(19) United States
(12) Patent Application Publication
(10) Pub. No.: US 2017/0348520 A1
(43) Pub. Date: Dec. 7, 2017

(54) ELECTROCEUTICAL DEVICE AND WRAP FOR USING THE SAME

(71) Applicant: 360 Approach to Health, LLC, Jupiter, FL (US)

(72) Inventors: Howard Busch, Jupiter, FL (US); Kevin Burch, West Palm Beach, FL (US); Susan Evans, Jupiter Island, FL (US); Gustav Karlsson, Tequesta, FL (US); Guenter Ginsberg, Bonita Springs, FL (US)

(21) Appl. No.: 15/682,769

(22) Filed: Aug. 22, 2017

Related U.S. Application Data
(63) Continuation of application No. PCT/US2017/021682, filed on Mar. 9, 2017.
(60) Provisional application No. 62/397,799, filed on Sep. 21, 2016.
(60) Provisional application No. 62/305,789, filed on Mar. 9, 2016.

(51) Int. Cl. A61N 1/04; A61N 1/36; A61N 7/00

(52) U.S. Cl. CPC A61N 1/0456; A61N 1/36171; A61N 1/36139; A61N 1/3615; A61N 1/36021; A61N 1/36167; A61N 2007/0017; A61N 2007/0021; A61N 2007/0026

(57) ABSTRACT

An electroceutical apparatus and self-care method for treating pain by providing Transcutaneous Electrical Nerve Stimulation (TENS) in combination with pulsed Ultrasound or Light Emitting Diode (LED) treatments. In some embodiments, the apparatus includes a pod unit and a controller, and wrap for holding the pod unit on an area of a user's body. In some embodiments, the wrap may include electrodes and the intensity of the TENS treatment may be adjusted by a user via the controller. In some embodiments, the frequency or output of the treatments is fixed and sequentially delivered to the user in a timed manner.

### I. RELATED APPLICATIONS

This application claims priority to U.S. Provisional Patent Application No. 62/305,789 entitled “Wrap for Electroceutical Device” filed on Mar. 9, 2016, and U.S. Provisional Patent Application No. 62/397,799 entitled “Electroceutical Device” filed on Sep. 21, 2016, all of which are incorporated herein by reference in their entireties.

### II. FIELD OF INVENTION

This disclosure relates to the self-care treatment of pain, both chronic and acute, using certain forms of energy. In certain embodiments, the disclosure describes an electroceutical device for non-invasive pain relief and/or accelerated healing self-care treatments.

### III. BACKGROUND

Physical pain is felt when nerves in the body communicate signals to the brain. One method of alleviating pain is to use electrical stimulation to block pain signals to the brain (i.e., essentially the brain receives a “busy signal” when a pain signal is trying to reach the brain). Treatments that use this method are generally referred to as electroceuticals. In general, electroceuticals, such as those available over-the-counter (OTC) are non-invasive devices that use certain forms of energy (referred to as modalities or therapies) to deliver specific actions that are of therapeutic benefit to the body.

Three primary, non-invasive and OTC electroceutical modalities are Transcutaneous Electrical Nerve Stimulation (TENS), Ultrasound (US), and Light Emitting Diodes (LEDs). TENS sends impulses to the nerves through electrodes that reduce or eliminate sensations or feelings of pain. These impulses override and/or block the normal pain signals being communicated to the brain. When the impulses are sent at particular frequencies, they may also help the body produce natural pain killers such as endorphins, which may prolong the pain relief sensation and aid in healing. Ultrasound generally uses deep, penetrating, high-frequency sound waves to deliver therapeutic benefits. A water based gel is applied to the skin under the ultrasonic device to conduct the sound waves into the body's tissues where it can be absorbed to reduce soft tissue inflammation, accelerate healing, improve range of motion, and decrease pain. LEDs produce particular wavelengths of light referred to as photons that penetrate the body's cells and deliver energy, stimulating cellular renewal and accelerating repair of damaged tissue. In particular, using wavelengths such as 660 nanometers (red light) and 880 nanometers (infrared light) promotes collagen and elastin for tissue growth, which helps accelerate healing and aids in pain reduction.

Conventional OTC electroceutical devices are typically limited to a single modality for treating pain and usually include a battery that needs to be replaced with use. OTC electroceutical devices that use a single modality can limit the pain relief and healing benefits possible from electroceuticals. Moreover, the effectiveness of a modality varies based on parameters such as frequency used and the area of the body treated. A user may not understand the best possible frequency or type of modality to use for treating an area of their body. Moreover, a user can experience trouble placing an OTC electroceutical device on an area of his or her body if the user has limited mobility, whether from a medical condition or otherwise. If fact, the pain that a user seeks to treat with the electroceutical device may itself limit physical dexterity.

Users can also experience problems properly placing the electrodes of an electroceutical device on an area of their body, as is conventionally manually done by the user. The proper placement of the electrodes in relation to the TENS device and to each other can significantly impact the effectiveness of the TENS treatment. Thus, a user can inadvertently limit the effectiveness of a TENS treatment by accidently placing the electrodes at improper locations. Moreover, since some conventional electrodes are attached by an adhesive, if a user attempts to change the location of the electrodes after they are attached, the user may experience discomfort (from the removal of the adhesive) and may have trouble reattaching the electrodes (since adhesives typically lose adhesive strength each time they are removed). After a limited number of uses, the electrodes must typically be replaced, even if they are otherwise functional.

Thus, there is a need in the art for an electroceutical device, especially an OTC electroceutical device, that can employ more than one modality of pain relief and/or accelerated healing to provide more effective pain relief and/or accelerated healing than currently available devices. There is a need for an OTC electroceutical device that can more easily be used by a user with limited mobility and that can take the guesswork out of the type and best settings for treatment, as well as the proper placement of the electroceutical device and electrodes on an area of the user's body. Finally, there is a need for an OTC electroceutical device that requires only a one-time purchase for use and eliminates the costs of replacement batteries for the device and/or the costs of replacement electrodes.

### IV. SUMMARY OF THE INVENTION

A method and apparatus for self-care of using non-invasive modalities to treat pain, such as musculoskeletal pain, and/or accelerate healing at selected areas of a user's body. In one embodiment, the apparatus comprises a controller and a pod unit. The controller and pod unit can be configured to be used by a layperson (i.e., not having medical training, knowledge, or experience) without the supervision of someone having medical training, knowledge, or experience. The controller and/or pod unit can be any color, such as white, or include a print or other design. Additionally, the controller and/or pod unit can include a brand, such as “360 Approach to Health.” The pod unit may be attached to an area of the body to be treated and may employ a TENS modality or a combination of TENS and LED or Ultrasound modalities to effect such treatment. In one embodiment, the combination of selected modalities have parameters that are preprogrammed into the apparatus and that, when in use, automatically deliver fixed frequencies or outputs in a sequential and timed manner to treat a specific area of a user's body. The apparatus can be portable. For example, the pod unit may be worn comfortably under clothing, weigh about 0.35 lbs and have a height of about 2.5 inches, width of about 1.5 inches, and thickness of about 1 inch. The controller can also be lightweight and be sized to fit into and be used by a user's hand. For example, the controller, can be of a rectangular shape having a height of about 4.5 inches, width of about 2.25 inches, and thickness of about 1 inch. The controller and pod unit can be of other shapes and sizes as well.

In one embodiment, the pod unit is “hands-free” and is held in place on an area of the user's body by a premade wrap. The wrap can be any color, such as black, or include a print or other design. Additionally, the wrap can include a brand, such as “360 Approach to Health.” The wrap can include a shaped hole for receiving a portion of the pod unit. The shaped hole may maintain the pod unit in place relative to the wrap using tension, friction, by interlocking features, by fasteners (such as snap fasteners), or otherwise. The wrap may also include electrodes that can be configured to removably couple with the pod unit (e.g., via wires or cables) for delivery of the treatment from the pod unit to an area of the user's body. One side of the electrodes (the side facing the user's skin) may include a layer of electrically-conductive gel to help facilitate current flow into the body. In one embodiment, the other side of the electrodes (the side facing the wrap) may include fastener(s) such as fabric hook and loop fasteners and/or snap fasteners for attaching the electrodes to the wrap via, for example, counterpart hook and loop fasteners and/or snap fastener receptacles affixed to the wrap on the side facing the user. The location of the counterpart fasteners on the wrap may be at any distance transversely away from the coupled pod unit and each other that ensures a therapeutically-desirable flow of electric current between the coupled electrodes. Thus, when the wrap is coupled to an area of the body, the electrodes and pod unit are also coupled to the area of the body without requiring an adhesive. Attaching electrodes, in particular, to a user's skin using an adhesive has many disadvantages such as pain and/or discomfort when removing the electrodes and the inability to reattach the electrodes to the body after a limited number of uses. When attached to a wrap by fastener(s) such as a fabric hook and loop fasteners and/or snap fasteners, the electrodes may be removed and reattached to a user's skin any number of times via the wrap. In one embodiment, the electrodes may be embedded in the wrap at a desirable distance away from a coupled pod unit and from each other to ensure a therapeutically-desirable flow of current between the electrodes. In one embodiment, a user is not required to guess where to place the electrodes relative to the pod unit or to each other.

In one embodiment, the wrap is sized and designed to comfortably accommodate a specific area of the body (e.g., back, hip, neck, shoulder, knee, elbow, wrist, ankle, or calf) by having, for example, curved corners and a thin shape to fit under clothing without drawing attention. The wrap may be adjustable by employing, for example, one or more straps with fasteners, such as mechanical-based fasteners, for example, fabric hook and loop fasteners and/or snap fasteners. The straps can be formed into the wrap, providing a unitary construction, or can be separate elements. The wrap and/or strap(s) (or portions of them) may be made of any useful material such as, e.g., plastic, vinyl, rubber, or cloth such as a cotton or polyester based cloth, and may employ an elastic material such as, e.g., neoprene.

In one embodiment, the controller can be configured to removably couple to the pod unit for controlling delivery of therapeutic care through the pod unit to an area of the user's body and/or to provide power to the pod unit. The controller can be configured to removably couple to the pod unit through a universal serial bus (USB) connection, a male to male TRRS (Tip-Ring-Ring-Sleeve) cable, or otherwise, including through a wireless connection. The pod unit and/or controller can include a rechargeable battery such as, for example, a 3.7 Volt, 1000 milliampere per hour lithium-ion polymer rechargeable battery having protection circuitry. To recharge the battery(ies), the controller may be configured to include a port for receiving a cable of a wall charger, such as a five Volt, two Ampere output wall charger.

The controller can receive an indication to begin the therapeutic treatment, for example from a user input, detect a type of therapeutic care to be delivered, for example based on the type of coupled pod unit, and communicate to the pod unit the type of therapeutic care to be delivered. In one embodiment, the type of delivered therapeutic care can adjust between TENS-based and LED-or Ultrasound-based therapeutic care and the intensity level of the TENS-based treatment can adjust between, for example, approximately 15 volts, 21 volts, 27 volts, 33 volts, 39 volts and 45 volts. The intensity of TENS-based therapeutic care may be based on inputs received from a user, for example, though an interface on the controller communicated to the pod unit. The controller may also communicate the intensity of the TENS-based therapeutic care and/or the type of therapeutic care being delivered at a particular moment in time to a user by, for example, activating one or more LEDs positioned on the controller or by making an audible “ring” or other sound. The controller may also communicate other information to the user through, for example, LEDs, such as whether the pod unit and/or controller is on or off, whether the pod unit is coupled to the controller, whether the pod unit and/or controller is charging or charged, and whether the pod unit and/or controller have a low battery.

In one embodiment of the invention, the apparatus is configured to provide therapeutic treatment to a specific area of the body. Examples of such areas include lower back, hip, neck, shoulder, knee, elbow, ankle, wrist, and calf. The below combinations are merely illustrative and are not intended to be exhaustive, limiting, or preferable.

When configured to treat the lower back or hip, the apparatus may use a TENS pulse with a frequency of about 10-40 Hertz (Hz) for 10 minutes followed with pulsed Ultrasound treatment at a frequency of about 1 megahertz (MHz) for about 10 minutes, followed by another TENS treatment with a frequency of about 60-100 Hz. The timing and frequency or output delivered to the user's lower hip or back during these three 10 minute periods are fixed (i.e., not changeable by the user) and programmed to run sequentially. The low TENS frequency is intended to activate the body's endorphins and provide pain relief. It is generally effective against chronic pain and has a significant carry over period of pain relief. The TENS high frequencies are responsible for pain-gate activation which is when transmission of the perception of pain to the brain is blocked by the activity of the large diameter, fast-conducting sensory nerve fibers. This activity effectively closes the gateway to the brain through which you would normally perceive pain. The TENS treatment may be used sequentially with pulsed Ultrasound to effectively penetrate tissues, which facilitates healing of the soft tissue.

When configured to treat the neck, the apparatus may use a TENS pulse with a frequency of about 10-40 Hz for 10 minutes followed with LED treatment at about 660 nanometers (red light) and/or about 880 nanometers (infrared light) for 10 minutes, followed by another TENS treatment with a frequency of about 60-100 Hz. The timing and frequency or output delivered to the user's neck during these three 10 minute periods are fixed (i.e., not changeable by the user) and programmed to run sequentially. The low TENS frequency may activate the body's endorphins and provide pain relief. It is generally effective against chronic pain and has a significant carry over period of pain relief. The TENS treatment may be used sequentially with LED treatment to release free radicals (considered the body's natural vasodilator), which allows for increased blood circulation to promote tissue healing.

When configured to treat the knee, the apparatus may use a TENS pulse with a frequency of about 60-100 Hz for 10 minutes followed with LED treatment at about 660 nanometers (red light) and/or about 880 nanometers (infrared light) for 10 minutes, followed by another TENS treatment with a frequency of about 60-100 Hz. The timing and frequency or output delivered to the user's knee during these three 10 minute periods are fixed (i.e., not changeable by the user) and programmed to run sequentially. A mid-range to high TENS frequency may be used for the knee because high frequencies effectively treat boney and superficial soft tissues, which are predominant in the knee; and mid-range frequencies block pain signals. The TENS mid to high frequencies are responsible for pain-gate activation which is when transmission of the perception of pain to the brain is blocked by the activity of the large diameter, fast-conducting sensory nerve fibers. This activity effectively closes the gateway to the brain through which you would normally perceive pain. The TENS treatment may be used sequentially with LED treatment to release free radicals (considered the body's natural vasodilator), which allows for increased blood circulation to promote healing.

When configured to treat the elbow, the apparatus may use a TENS pulse with a frequency of about 60-100 Hz for 10 minutes followed with Ultrasound treatment at a frequency of about 1 MHz for 10 minutes, followed by another TENS treatment with a frequency of about 60-100 Hz. The timing and frequency or output delivered to the user's elbow during these three 10 minute periods are fixed (i.e., not changeable by the user) and programmed to run sequentially. A mid-range to high TENS frequency may be used for the elbow because high frequencies effectively treat boney and superficial soft tissues, which are predominant in the elbow; and mid-range frequencies block pain signals. The TENS mid to high frequencies are responsible for pain-gate activation which is when transmission of the perception of pain to the brain is blocked by the activity of the large diameter, fast-conducting sensory nerve fibers. This activity effectively closes the gateway to the brain through which you would normally perceive pain. The TENS treatment may be used sequentially with pulsed Ultrasound to effectively penetrate tissues, which facilitates healing of the soft tissue.

When configured to treat the shoulder, the apparatus may use a TENS pulse with a frequency of about 20-80 Hz for 10 minutes followed with Ultrasound treatment at a frequency of about 1 MHz for 10 minutes, followed by another TENS treatment with a frequency of about 20-80 Hz. The timing and frequency or output delivered to the user's shoulder during these three 10 minute periods are fixed (i.e., not changeable by the user) and programmed to run sequentially. A low to mid-range TENS frequency may be used for the shoulder and calf because low frequencies activate the body's endorphins and provide pain relief; mid-range frequencies block pain signals. The frequency range is effective against chronic pain and has a significant carry over period of pain relief. The TENS treatment may be used sequentially with pulsed Ultrasound to effectively penetrate tissues, which facilitates healing of the soft tissue.

When configured to treat the ankle or wrist, the apparatus may use a TENS pulse with a frequency of about 80-150 Hz for 10 minutes followed with LED treatment at about 660 nanometer (red light) and/or about 880 nanometers (infrared light) for 10 minutes, followed by another TENS treatment with a frequency of about 80-150 Hz. The timing and frequency or output delivered to the user's ankle or wrist during these three 10 minute periods are fixed (i.e., not changeable by the user) and programmed to run sequentially. A high TENS frequency may be used to treat boney and superficial soft tissues, which are predominant in the ankle or wrist. The TENS high frequencies are responsible for pain-gate activation which is when transmission of the perception of pain to the brain is blocked by the activity of the large diameter, fast-conducting sensory nerve fibers. This activity effectively closes the gateway to the brain through which you would normally perceive pain. The TENS treatment may be used sequentially with LED treatment to release free radicals (considered the body's natural vasodilator), which allows for increased blood circulation to promote healing.

When configured to treat the calf, the apparatus may use a TENS pulse with a frequency of about 20-80 Hz for 10 minutes followed with LED treatment at about 660 nanometers (red light) and/or about 880 nanometers (infrared light) for 10 minutes, followed by another TENS treatment with a frequency of about 20-80 Hz. The timing and frequency or output delivered to the user's calf during these three 10 minute periods are fixed (i.e., not changeable by the user) and programmed to run sequentially. A low to mid-range TENS frequency may be used for the calf because low frequencies activate the body's endorphins and provide pain relief; and mid-range frequencies block pain signals. The frequency range is effective against chronic pain and has a significant carry over period of pain relief. The TENS treatment may be used sequentially with LED treatment to release free radicals (considered the body's natural vasodilator), which allows for increased blood circulation to promote healing.

In one embodiment, the treatment (whether TENS and LED or TENS and Ultrasound or otherwise) is used in combination with other products such as creams or gels (for example, the commercial product known as “360 Turned Up”). When using a cream or gel, the cream or gel may be applied to the treatment area concurrently with apparatus application to further potentiate pain relief.

In one embodiment, the apparatus allows only a single, continuous mode of treatment at a time for a pre-programmed period of time. The mode may be non-adjustable other than intensity. Whether the embodiment is TENS and LED or TENS and Ultrasound, the treatments may run sequentially. In one embodiment, the intensity may vary between zero and 50 Volts into a 1000 Ohm load. As discussed below, the level of intensity of the TENS-based treatment may be selected by the user. The mode (including number of cycles) may last for a period of time and the TENS frequency may vary during the period, depending, for example, on the area of the body treated. Intensity of the Ultrasonic or LED output does not vary and cannot be adjusted by, for example, the user. To illustrate, when treating the lower back or hip, the mode may have two cycles, wherein the area may be treated with a low TENS frequency (about 10-40 Hz) for about the first 10 minutes and then treated with a mid-range to high TENS frequency (about 60-100 Hz) for about the last 10 minutes. The user may adjust the intensity of the TENS treatment delivered (within in a preprogrammed range) during these periods. Between these 10 minute TENS treatments, the area of the lower back or hip may be treated with pulsed Ultrasound for about 10 minutes wherein the output may be about 1 MHz for a 50% duty cycle at 0.25 Watts per centimeter squared. The intensity of the Ultrasound output may not be adjusted by the user.

When treating the neck, the mode may have two cycles, wherein the area may be treated with a low TENS frequency (about 10-40 Hz) for about the first 10 minutes and then treated with a mid-range to high TENS frequency (about 60-100 Hz) for about the last 10 minutes of the treatment. The user may adjust the intensity of the TENS treatment delivered (within in a preprogrammed range) during these periods. Between these 10 minute TENS treatments, the area of the neck may be treated with LEDs for about 10 minutes, wherein the output of the LEDs may be about 0.214 Joules (for 660 nanometer or red light) and about 22.4 milliwatts per square radian (for 880 nanometer or infrared light). The intensity of the LED output may not be adjusted by the user.

When treating the knee, the mode may have two cycles, wherein the area is treated with a mid-range to high TENS frequency (about 60-100 Hz) for about the first and last 10 minutes of the treatment. The user may adjust the intensity of the TENS treatment delivered (within in a preprogrammed range) during these periods. Between these 10 minute high-frequency TENS treatments (about 60-100 Hz), the area of the knee may be treated with LEDs for about 10 minutes, wherein the output of the LEDs may be about 0.214 Joules (for 660 nanometer or red light) and about 22.4 milliwatts per square radian (for 880 nanometer or infrared light). The intensity of the LED output may not be adjusted by the user.

When treating the elbow, the mode may have two cycles, wherein the area may be treated with a mid-range to high TENS frequency (about 60-100 Hz) for about the first and last 10 minutes of the treatment. The user may adjust the intensity of the TENS treatment delivered (within in a preprogrammed range) during these periods. Between these 10 minute TENS treatments, the area of the elbow may be treated with pulsed Ultrasound for about 10 minutes, wherein the output may be about 1 Megahertz for a 50% duty cycle at 0.25 Watts per centimeter squared. The intensity of the Ultrasound output may not be adjusted by the user.

When treating the shoulder, the mode may have two cycles, wherein the area may be treated with a low to mid-range TENS frequency (about 20-80 Hz) for about the first and last 10 minutes of the treatment. The user may adjust the intensity of the TENS treatment delivered (within in a preprogrammed range) during these periods. Between these 10 minute TENS treatments, the area of the shoulder or calf may be treated with pulsed Ultrasound for about 10 minutes, wherein the output may be about 1 MHz for a 50% duty cycle at 0.25 Watts per centimeter squared. The intensity of the Ultrasound output may not be adjusted by the user.

When treating the ankle or wrist, the mode may have two cycles, wherein the area may be treated with a high TENS frequency (about 80-150 Hz) for about the first and last 10 minutes of the treatment. The user may adjust the intensity of the TENS treatment delivered (within in a preprogrammed range) during these periods. Between these 10 minute TENS treatments, the area of the ankle or wrist may be treated with LEDs for about 10 minutes, wherein the output of the LEDs may be about 0.214 Joules (for 660 nanometer or red light) and about 22.4 milliwatts per square radian (for 880 nanometer or infrared light). The intensity of the LED output may not be adjusted by the user.

When treating the calf, the mode may have two cycles, wherein the area may be treated with a low to mid-range TENS frequency (about 20-80 Hz) for about the first and last 10 minutes of the treatment. The user may adjust the intensity of the TENS treatment delivered (within in a preprogrammed range) during these periods. Between these 10 minute TENS treatments, the area of the calf may be treated with LEDs for about 10 minutes, wherein the output of the LEDs may be about 0.214 Joules (for 660 nanometer or red light) and about 22.4 milliwatts per square radian (for 880 nanometer or infrared light). The intensity of the LED output may not be adjusted by the user.

The foregoing has outlined rather broadly certain features and technical advantages of embodiments of the present invention in order that the detailed description that follows may be better understood. Additional features and advantages will be described hereinafter that form the subject of the claims of the invention. It should be appreciated by those having ordinary skill in the art that the conception and specific embodiment disclosed may be readily utilized as a basis for modifying or designing other structures for carrying out the same or similar purposes. It should also be realized by those having ordinary skill in the art that such equivalent constructions do not depart from the spirit and scope of the invention as set forth in the appended claims. Additional features will be better understood from the following description when considered in connection with the accompanying figures. It is to be expressly understood, however, that each of the figures is provided for the purpose of illustration and description only and is not intended to limit the present invention.

### V. BRIEF DESCRIPTION OF THE DRAWINGS

The following drawings illustrate by way of example not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiment depicted in the figure.

FIG. 1a is a front view (side facing away from a user's skin) of a portion of a wrap according to one embodiment of the present invention.

FIG. 1b (not to scale) is a front view (side facing away from a user's skin) of a strap according to one embodiment of the present invention.

FIG. 1c is a front view (side facing away from a user's skin) of a portion of a wrap according to one embodiment of the present invention that is different than the embodiment depicted in FIG. 1a.

FIG. 2a is a front view (side facing away from a user's skin) of a portion of a wrap according to one embodiment of the present invention that is different than the embodiment depicted in FIGS. 1a and 1c.

FIG. 2b is a bottom view (side facing a user's skin) of the wrap depicted in FIG. 2a according to one embodiment of the present invention.

FIG. 2c is a front view of a wrap according to one embodiment of the present invention that is different than the embodiment of FIG. 2a.

FIG. 3a is a top view (side facing away from the user's skin) of electrodes according to one embodiment of the present invention.

FIG. 3b1 is a top view (side facing away from the user's skin) of electrodes according to one embodiment of the present invention that is different than the embodiment depicted in FIG. 3a.

FIG. 3b2 is a side view of the electrodes depicted in FIG. 4b1 according to one embodiment of the present invention.

FIG. 4a1 is a top view (side facing away from the user's skin) of a pod unit according to one embodiment of the present invention.

FIG. 4a2 (not to scale) is a side view of the pod unit of FIG. 4a1 according to one embodiment of the present invention.

FIG. 4a3 is a bottom view (side facing the user's skin) of the pod unit of FIG. 4a1 according to one embodiment of the present invention.

FIG. 4b1 is a top view (side facing away from the user's skin) of a pod unit according to an embodiment of the present invention that is different than the embodiment depicted in FIG. 4a1.

FIG. 4b2 is a side view of the pod unit of FIG. 4b1 according to one embodiment of the present invention.

FIG. 4b3 is a bottom view (side facing the user's skin) of the pod unit of FIG. 4b1 according to one embodiment of the present invention.

FIG. 4b3a is a bottom view (side facing the user's skin) of a pod unit according to one embodiment of the invention, wherein the pod unit comprises Ultrasound components.

FIG. 4b3b is a bottom view (side facing the user's skin) of a pod unit according to one embodiment of the invention, wherein the pod unit comprises LED components.

FIG. 4b4 is a partial cross-sectional view of the pod unit of FIG. 4b1 according to one embodiment of the present invention.

FIG. 5a is perspective view of the controller according to one embodiment of the present invention.

FIG. 5b is a perspective view of the controller according to another embodiment of the present invention.

FIG. 5c is a perspective view of the controller according to yet another embodiment of the present invention.

FIG. 6a is an electronic block diagram of the controller of one embodiment of the present invention, such as that shown in FIG. 5a, 5b, or 5c.

FIG. 6b is a flow chart describing operation of the controller of FIG. 6a when coupled to a pod unit according to one embodiment of the present invention.

FIG. 7 is a plan view of an electroceutical treatment system (side facing away from the user's skin) according to one embodiment of the present invention.

FIG. 8 is a bottom view (side facing user's skin) of a portion of an electroceutical treatment system according to one embodiment of the present invention.

### VI. DETAILED DESCRIPTION OF EMBODIMENTS OF THE PRESENT INVENTION

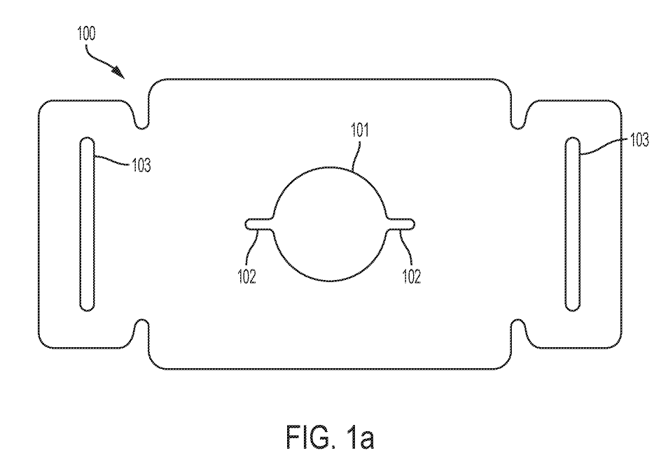

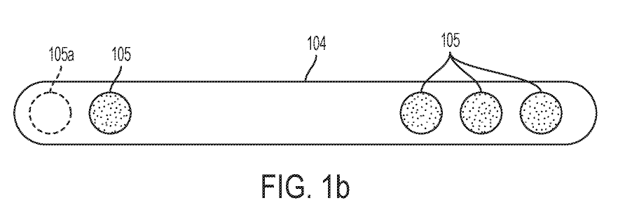

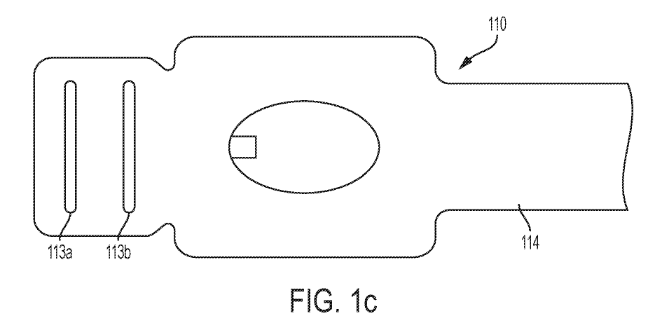

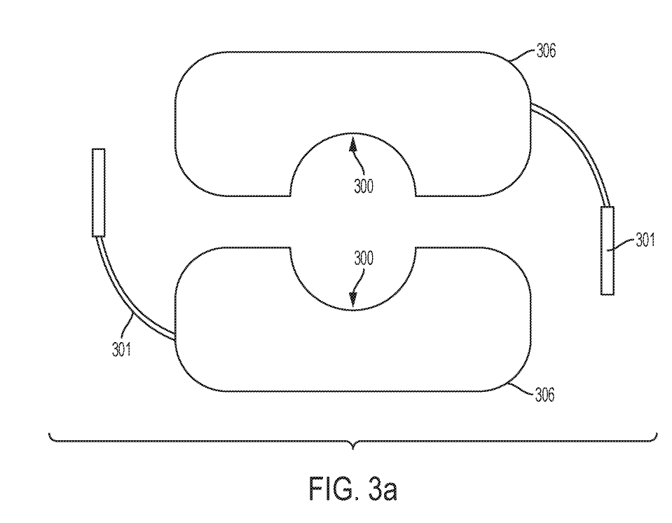

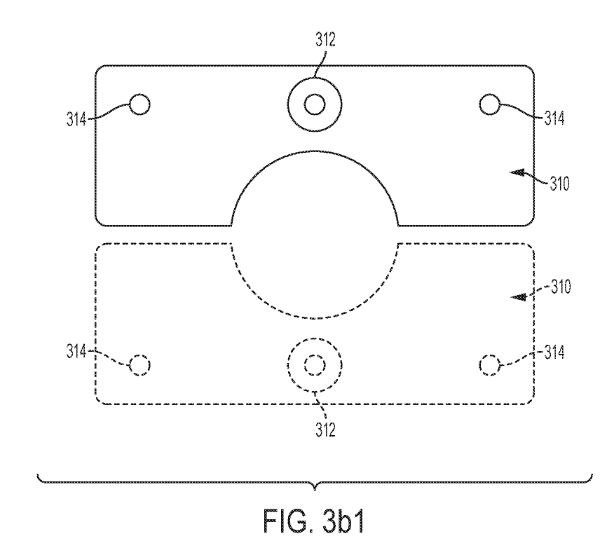

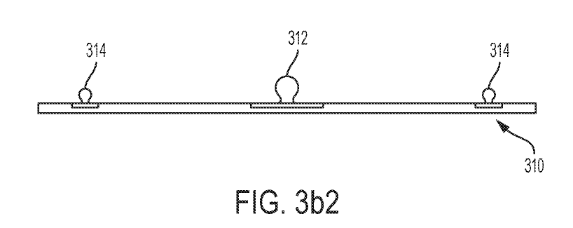

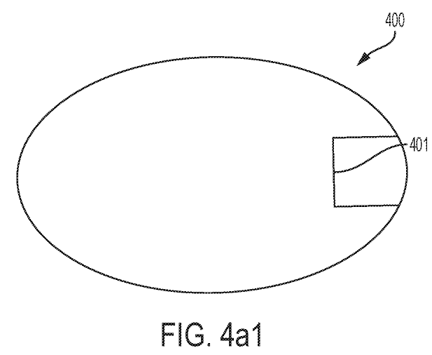

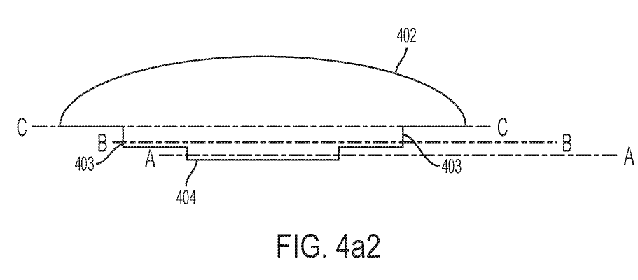

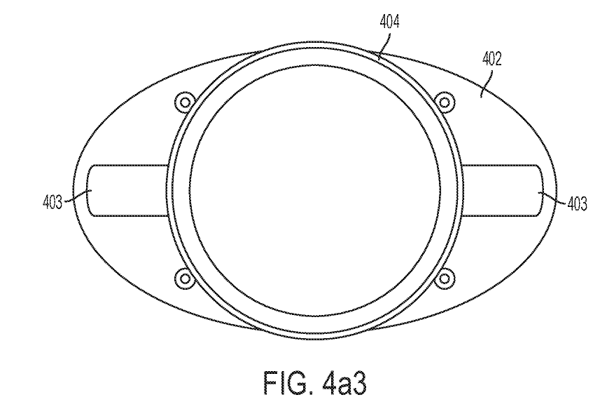

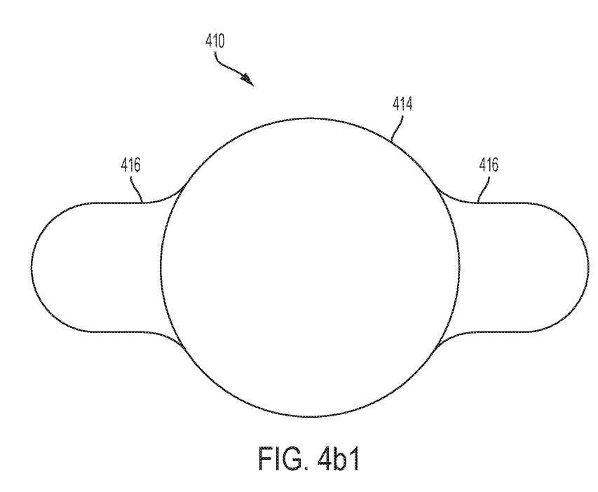

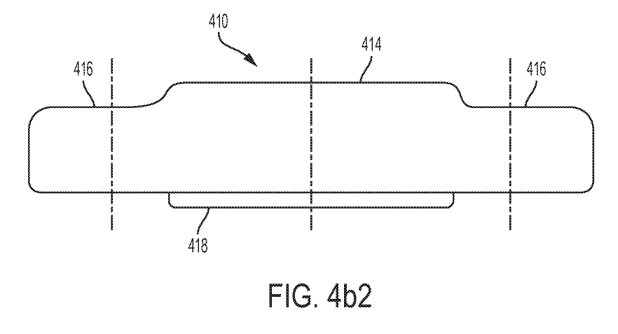

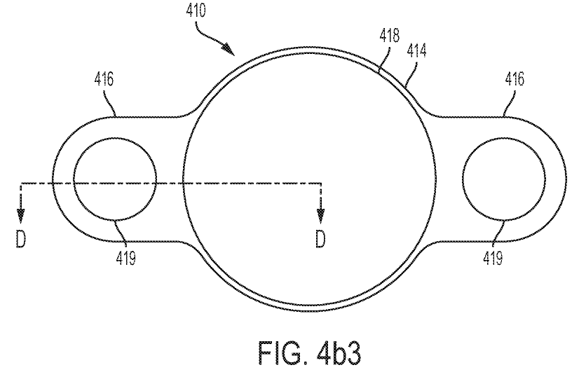

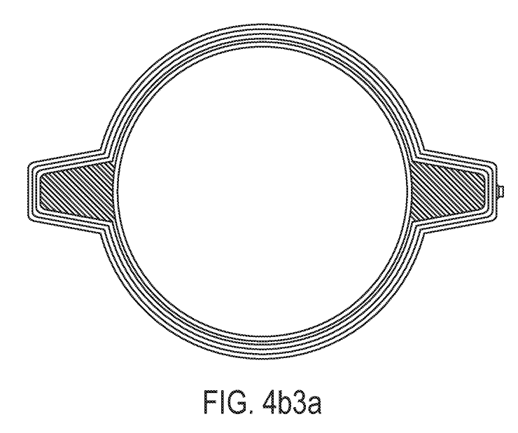

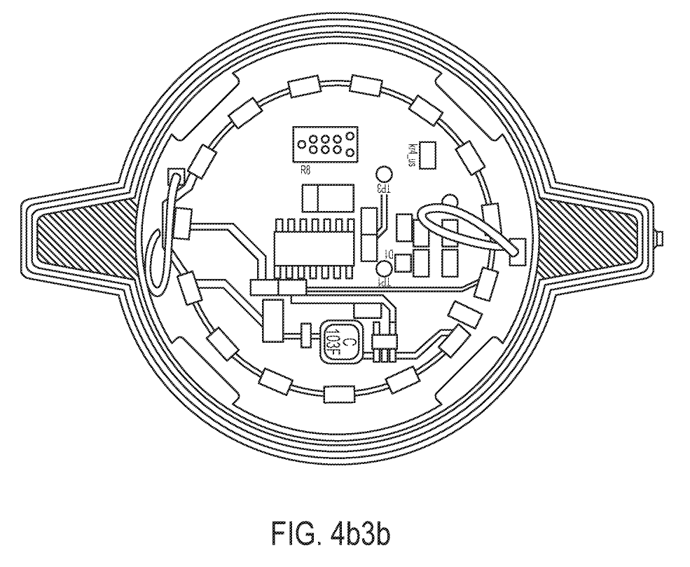

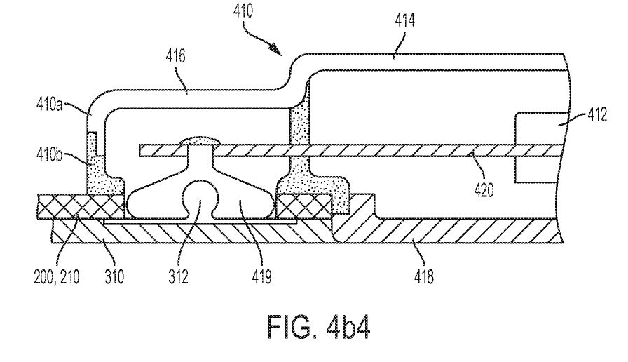

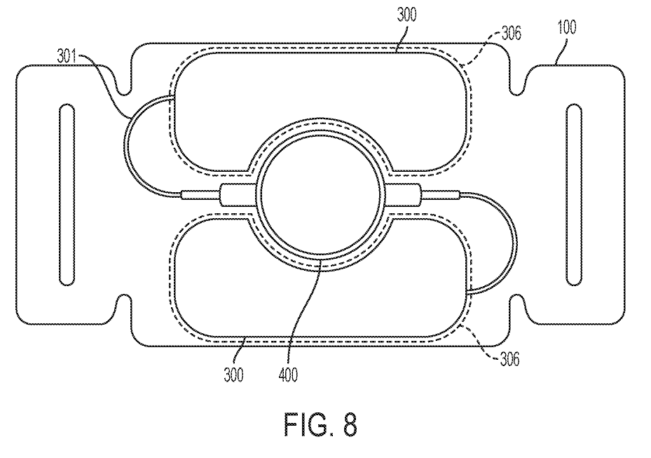

FIG. 1a shows a portion of one embodiment of the wrap 100 with an ergonomic shape and a thickness less than its length or width, so that it may comfortably be worn by a user underneath clothing. Wrap 100 can be made of neoprene or another comfortable material. Wrap 100 can include electrode fasteners 307 (see FIG. 8) on it bottom side (side facing user's skin) that can removably (i.e., without requiring a tool) attached to (and roughly fit the shape of) electrodes 300 (see FIG. 3a). The wrap 100 has a hole 101 including a plurality of slits 102. A pod unit 400 (see FIG. 4a1) can be removably coupled to wrap 100 via, for example, hole 101 and slits 102. Wrap 100 also includes a plurality of slots 103 near its ends for attaching a plurality of straps 104 (see FIG. 1b).

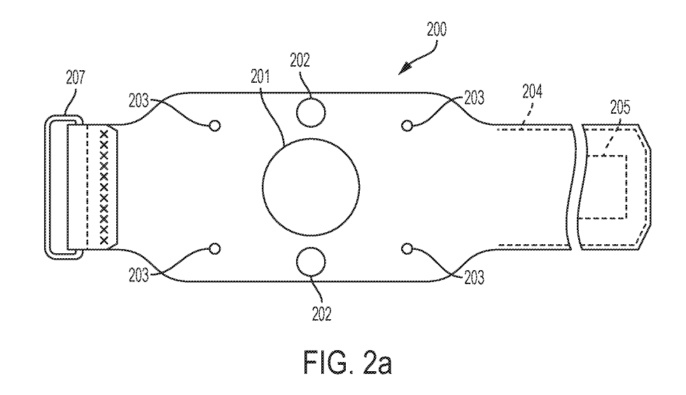

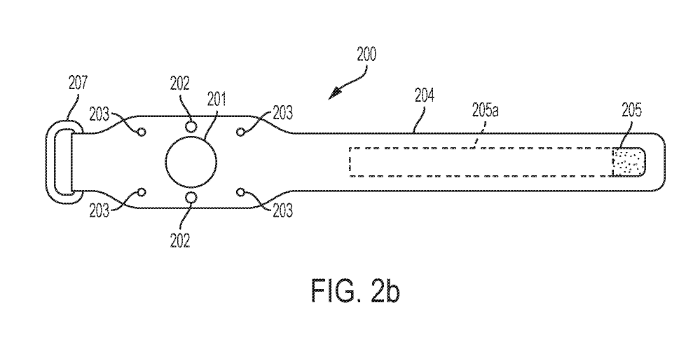

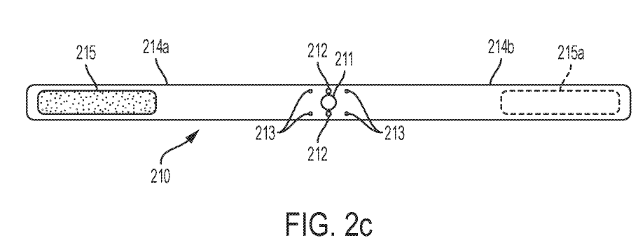

Straps 104, shown in FIG. 1b, may have a thickness less than their length or width, so that they may be comfortably worn underneath clothing. The strap may be, for example, 1-5 mm thick. The front of strap 104 (side facing away from a user's skin) includes one or more strap fasteners 105 located closer to one end of strap 104. Alternatively, strap fasteners 105 may comprise a single, unitary strip having sufficient length to allow adjustment. Strap fasteners 105 may be of any type conventionally known, such as button fasteners, snap fasteners, or fabric hook and loop fasteners or may be a type later-invented. Strap 104 also includes one or more wrap fasteners 105a (relative location shown in dotted line) on its bottom side, positioned close to the end of strap 104 opposite that of the plurality of strap fasteners 105. Strap 104 can be attached to wrap 100 by feeding its end closest to wrap fastener 105a through a slot 103 of wrap 100 and attaching wrap fastener 105a to a strap fastener 105, such as the strap fastener closest to wrap fastener 105a. Such attachment may be accomplished in any conventional or later developed manner. For example, when using a fabric hook and loop fastener, as illustrated here, strap fasteners 105 could be the “hook” side of the fabric hook and loop fastener and wrap fastener 105a could be the “loop” side, or vice versa. Once attached, wrap fastener 105a and its corresponding strap fastener 105 are strong enough in the transverse direction, i.e., the direction of force when the strap is pulled, to reasonably resist detachment. A second strap (not shown) is generally attached to another slot 103 in the same manner as first strap 104. The second strap may be identical to first strap 104, except that its strap fasteners contain counterpart fasteners corresponding to the strap fasteners of first strap 104. For example, if strap fasteners 105 are fabric hook and loop fasteners, the strap fasteners on strap 104 could be the “hook” side of the fabric hook and loop fastener and the strap fasteners on the second strap could be the “loop” side of the fabric hook and loop fastener, or vice versa. As another example, if strap fasteners 105 on strap 104 are snap fasteners, the strap fasteners on the second strap can be snap fastener receptacles, such as snap fastener receptacles 203, shown in FIG. 2 In these ways, the two straps may be fastened to one another after wrapping around a portion of the user's body by attaching the strap fasteners of strap 104 to the counterpart strap fasteners of the second strap.

FIG. 1c depicts a portion of a wrap 110 according to an embodiment of the present invention having a pod unit removably coupled thereto. Wrap 110 has similar features as wrap 100 except that strap 114, unlike strap 104, is included as part of wrap 110. Strap 114 does not include strap fasteners 105 and does not attach to wrap 110 or another strap by fasteners. Instead, strap 214 may have a length sufficient to reach around an area of the body and then to attach to an end of wrap 110 using, for example, slots 113a, 113b. Slots 113a, 113b are on the same end of wrap 110 and facilitate connection by strap 114 by feeding strap 114, for example, first through slot 113a from the side facing away from the user's skin to the side facing the user's skin, and then through slot 113b from the side facing the user's skin to the side facing away from the user's skin, or vice versa. The strap can be held in place by tension, friction, or otherwise, and may optionally be fed back through one of slots 113a, 113b to more firmly hold the wrap in place. To facilitate firm placement of the strap by tension and/or friction, the width (i.e., the dimension running parallel with the length of strap 114) of slots 113a, 113b may be only slightly larger than the thickness of strap 114. Additionally, wrap 110, including strap 114, may be made from a material having a large coefficient of friction, such as neoprene.

In another embodiment (not shown), a single slot may be disposed in an end of the wrap 110 and strap 114 may include one or more strap fasteners on one side, e.g., its top, close to its free end, i.e., the end spaced apart from the hole for the pod unit, and counterpart strap fasteners on the same side, e.g., its top, but disposed relatives further away from its free end, i.e., closer to hole for the pod unit, such that strap 114 may be fed through one end of the single slot, e.g., from the top side of strap 114 to the bottom side of strap 114, and folded back over itself to removably couple the strap fasteners and counterpart strap fasteners together. In one embodiment, the strap fasteners and counterpart strap fasteners may comprise a single, unitary piece that is of sufficient length to permit adjustment of wrap 110.

FIG. 2a depicts a front view (side facing away from a user' skin) of a portion of a wrap 200 according to an embodiment of the present invention. Wrap 200 has an ergonomic shape and a thickness less than its length or width, so that it may comfortably be worn by a user underneath clothing. For example, wrap 200 may be about 2 mm thick and made of neoprene. Wrap 200 includes a central hole 201 for receiving a portion of a pod unit, such as pod unit 410 (see FIGS. 4b1-4b4). Wrap 200 also includes secondary holes 202 configured to allow electrodes, such as electrodes 310 (see FIGS. 3b1-3b2), to removably couple to the pod unit through the wrap, as shown in FIG. 4b4, such as via large snap fasteners and large snap fastener receptacles. Wrap 200 also includes snap fastener receptacles 203 configured to removably couple to correspondingly sized snap fasteners, such as snap fasteners 314 of electrodes 310. Snap fastener receptacles 203 can made from plastic or another material.

A strap 204 is included as part of wrap 200 on one end of wrap 200. The other end of wrap 200 includes a buckle 207 that can be made from steel or another rigid material. As shown more clearly in FIG. 2b, which is a back view (side facing a user's skin) of wrap 200, strap 204 includes one or more strap fasteners 205, such as hook and loop fasteners on the end spaced apart from the central portion of the wrap (i.e., away from the portion having central hole 201). The other side of strap 204 can include corresponding strap fasteners 205a (relative location shown in dotted line). For example, strap fasteners 205 could be the “hook” side of a fabric hook and loop fastener and corresponding strap fasteners 205a could be the “loop” side of a fabric hook and loop fastener, or vice versa. To couple wrap 200 to a portion of a user's body, strap 204 is wrapped around the portion of the user's body (e.g., arm, leg), fed through buckle 207, and then back over itself so that strap fasteners 205 removably couple to corresponding strap fasteners 205a. Corresponding strap fasteners 205a can be disposed along most of the length of strap 204 on one side so that the length of strap 205 (and thus the length of wrap 200) can be adjusted.

FIG. 2c depict a front view (side facing away from a user's skin) of a wrap 210 according to one embodiment of the present invention that is similar to wrap 200 of FIG. 2a, but shaped to wrap around a larger portion of the user's body, such as the user's torso, so that a portion of the user's body, such as their back, may be treated by an electroceutical device coupled to the wrap 210. For example, wrap 210 may be about 50 inches in length. Wrap 210 has an ergonomic shape and a thickness less than its length or width, so that it may comfortably be worn by a user underneath clothing. For example, wrap 210 may be about 2 mm thick and made of neoprene. Wrap 210 include a central hole 211 that can receive a portion of a pod unit, such as pod unit 410 (see FIGS. 4b1-4b4). Wrap 210 also includes secondary holes 212 configured to allow electrodes, such as electrodes 310 (see FIGS. 3b1-3b2), to removably couple to the pod unit through the wrap, as shown in FIG. 4b4, such as via large snap fasteners and large snap fastener receptacles. Wrap 210 includes snap fastener receptacles 213 that may be removably coupled to snap fasteners of electrodes such as snap fasteners 314 of electrodes 310. Snap fastener receptacles 213 made from plastic or another material.

Wrap 210 includes two strap portions 214a and 214b. Strap portion 214a includes a strap fastener 215 on the side facing away from a user's skin. Strap fastener 215 can be any type of removable fastener such as a fabric hook and loop fastener or a snap fastener. Strap fastener 215 can be one continuous piece or can be separate, spaced apart pieces. Strap portion 214b includes a strap fastener 215a on the side facing a user's skin. Strap fastener 215a can be one continuous piece or can be separate, spaced apart pieces. Strap fastener 215a corresponds to and is configured to removably couple with strap fastener 215 of strap portion 214a. For example, strap fastener 215 can be a hook portion of fabric hook and loop and fastener and corresponding strap fastener 215a can be the loop portion of a fabric hook and loop fastener, or vice versa. As a further example, strap fastener 215 can be a snap fastener and corresponding strap fastener 215a can be a snap fastener receptacle, or vice versa. Strap fastener 215 and/or corresponding strap fastener 215a can be of sufficient length/number to adjust the length of wrap 210 when coupled together.

FIG. 3a depicts a front view (facing away from a user's skin) of two electrodes 300 according to an embodiment of the present invention. Electrodes 300 can be shaped to fit on the back side (facing user's skin) of a wrap, such as wraps 100 or 110 and around hole 101 to not interfere with a pod unit disposed therein. Electrodes 300 each include an electrode wire 301 with an end that may attach to the pins located within coverings 403 of a pod unit 400. The front side (side shown in FIG. 3a) of electrodes 300 includes a readily-detached (i.e., without requiring a tool) electrode fastener 306 such as a fabric hook and loop fastener that attaches to corresponding electrode fasteners 307 (see FIG. 8) on the back side (facing user's skin) of wrap 100 or wrap 110 in a conventional manner. For example, if using a fabric hook and loop fastener, electrode fasteners 306 could be the “hook” and electrode fasteners 307 could be the “loop,” or vice versa. The back side of electrodes 300 (facing a user's skin) may include a gel to better conduct electric current through a user's body.

FIG. 3b1 depicts a front view (facing away from a user's skin) of two electrodes 310 according to an embodiment of the present invention that is different than the embodiment depicted in FIG. 3a. Electrodes 310 can be shaped to fit on the back side (facing user's skin) of a wrap having snap fastener receptacles, such as snap fastener receptacles 203 or 213 of wrap 200, 210, respectively, around a central hole, such as hole 201 or hole 211 of wrap 200 or wrap 210, respectively, such that electrodes 310 do not interfere with the coupling of a pod unit, such as pod unit 410, to the wrap through the central hole. Electrodes 310 can include one or more snap fasteners 314 configured to removably couple the electrodes to the wrap via the snap fastener receptacles of the wrap. For example, electrodes 310 can be removably coupled to wrap 200 or wrap 210 by coupling snap fasteners 314 with snap fastener receptacles 203 or 213, respectively. Snap fasteners 314 can be made from a non-conductive material such as plastic.

Electrodes 310 can include one or more large snap fasteners 312 configured to removably couple the electrodes to the pod unit via large snap fastener receptacles on the pod unit, such as large snap fastener receptacles 419 of pod unit 410 (see FIG. 4b4). As partially shown in FIG. 4b4, electrodes 310 can be advantageously coupled to one side of a wrap, such as wrap 200 or wrap 210, via snap fasteners 314 and snap fastener receptacles 203 or 213, respectively, and also to a pod unit, such as pod unit 410 via large snap fasteners 312 and large snap fasteners receptacles 419, on the other side of the wrap via secondary holes of the wrap, such as secondary holes 202 or 212 of wrap 200 or 210, respectively. In this configuration, the pod unit is functionally coupled to the wrap. Large snap fasteners 312 can be made from a conductive material such as metal.

Electrodes 310 also include embedded wire(s) (not shown) that conduct electric current from large snap fasteners 312 into electrodes 310, for example from large snap fastener receptacles 419 (when made of an electrically conductive material such as metal) of pod unit 410 through coupled large snap fasteners 312. FIG. 3b2 depicts a side view of electrodes 310 of FIG. 3b1.

FIG. 4a1 depicts a top view (facing away from the user's skin) of an embodiment of the pod unit 400. As shown, the pod unit may include a port 401 for communicating with a handheld controller for various functions such as turning on and off pod unit 400, adjusting intensity of electrodes, and monitoring a treatment. Pod unit 400 can perform TENS treatment alone or in conjunction (although not necessarily temporally) with other electroceutical treatments such as Ultrasound or LED treatment and can be configured to provide treatment to a specific area of the body (e.g., lower back, hip, neck, shoulder, knee, elbow, ankle, wrist, and/or calf), as illustratively explained above. Pod unit 400 can be made of any suitable materials, such as lightweight plastics. Pod unit 400 can have an oval shape as shown in FIG. 4a1 with a length greater than its width and thickness, such that when positioned in a wrap, such as wrap 100 or wrap 110, it can be worn comfortably under clothing. For example, pod unit 400 can have a length of 5 centimeters and a thickness of 1.5 centimeters.

FIG. 4a2 depicts a side view of one embodiment of the pod unit 400 of FIG. 4a1. As seen in FIG. 4a2, pod unit 400 includes a primary portion 402 that can house TENS treatment components and/or other treatment components such as LED or Ultrasound components. The TENS treatment components may be connected to electrodes through coverings 403. Coverings 403 house pins (not shown) that facilitate electric connection with electrode wires, such as electrode wires 301 shown in FIG. 3a. Pod unit 400 includes a secondary portion 404 that can house other electroceutical treatment components such as an Ultrasound or LED treatment components. The cross-sectional area along line A-A of secondary portion 404 and line B-B of coverings 403 can be slightly larger than the dimensions of hole 101 and slits 102, respectively, of wrap 100 or wrap 110 so that pod unit 400 can fit snugly in hole 101 of wrap 100 or wrap 110 and be held in place by tension and/or friction. The cross-sectional area of primary portion 402 along line C-C can be larger than hole 101 of wrap 100 or wrap 110 to prevent the pod unit 400 from passing through hole 101. In operation, pod unit 400 is inserted into the front side (facing away from the user's skin) of wrap 100 or wrap 110 through hole 101 with secondary portion 404 entering hole 101 first.

FIG. 4a3 depicts a bottom view (facing user's skin) of one embodiment of pod unit 400 employing TENS treatment components in primary housing 402 and other electroceutical components such as Ultrasound or LED components in secondary housing 404. Pod unit 400 can be connected to electrodes 300 via pins (not shown) positioned in coverings 403, for example, after coupling to wrap 100 or wrap 110, as just described. Secondary portion 404 can extend far enough to be in contact with a portion of a user's skin when pod unit 400 is coupled to a wrap, such as wrap 100 or wrap 110.

In one embodiment of pod unit 400 (not depicted), the top surfaces of coverings 403 (i.e., the side facing away from a user's skin) do not physically contact the bottom surfaces of primary portion 402. In such an embodiment, coverings 403 directly physically contact only the side of secondary portion 404. The gap between the top surface of coverings 403 and the bottom surface of primary portion 402 can be about the thickness of wrap 100 or wrap 110 and allow the pod unit 400 to not only fit snugly within hole 101 but to be thereafter twisted to interlock with wrap 100 or wrap 110 by positioning coverings 403 at a different planar location than slits 102. In such an embodiment, pod unit 400 can be removably coupled to wrap 100 or wrap 110 by the interlocking just described as well by tension and/or friction.

FIG. 4b1 depicts a top view of pod unit 410 according to an embodiment of the present invention. Pod unit 410 has the same or similar functionality as pod unit 400 of FIG. 4a1 but different physical features, including a different physical shape, that can also be worn comfortably under clothing. Pod unit 410 includes a port 412 (a portion of which is shown in FIG. 4b4) for communicating with a handheld controller for various functions such as turning on and off pod unit 410, adjusting intensity of electrodes, and monitoring a treatment. Pod unit 410 also includes a central portion 414 for housing TENS treatment components and/or other treatment components such as LED or Ultrasound components, and a plurality of flange portions 416 for housing electrode interfaces. Central portion 414 and flange portions 416 may be a single unitary piece or may be made of separate, interlocking pieces. FIG. 4b2 depicts a side view of the pod unit 410 of FIG. 4b1. As shown, pod unit 410 includes a cover 418 that can be made of a material capable of transmitting Ultrasound or LED outputs, such as stainless steel (see FIG. 4b3a) or plastic (see FIG. 4b3b), respectively. Cover 418 can extend from the bottom of flange portions 416 far enough to contact an area of a user's skin when the pod unit is coupled to, for example, wrap 200 or wrap 210. FIG. 4b3 depicts a bottom view of the pod unit 410. Large snap receptacles 419, shown more clearly in FIG. 4b4, can be located in peripheral portions 414 and can be removably coupled to, for example, large snaps 312 on wrap electrodes 310 (see FIGS. 3b1 and 3b2). Large snap receptacles 419 can be made from a similar material as large snaps 312, i.e., conductive metal, such that an electric current can pass therethrough.

FIG. 4b4 depicts a cross-sectional view of pod unit 410 along the line D-D of FIG. 4b3 when pod unit 410 is coupled to a wrap such as, for example, wrap 200 or wrap 210 and to electrodes, such electrodes 310. As shown, pod unit 410 can include a top housing portion 410a and a bottom housing portion 410b can that be fixedly or removably coupled together by, for example, screws or plastic bonding. Pod unit 410 includes an interface 420, such as a printed circuit board (PCB), fixedly coupled to a large snap receptacle 419, which is removably coupled to a large snap fastener 312 such that an electric current can flow from interface 420 through large snap receptacle 419 and large snap fastener 312 and into electrodes 310.

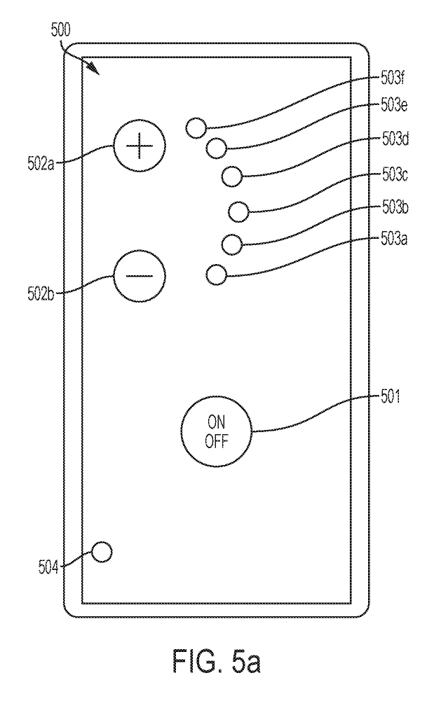

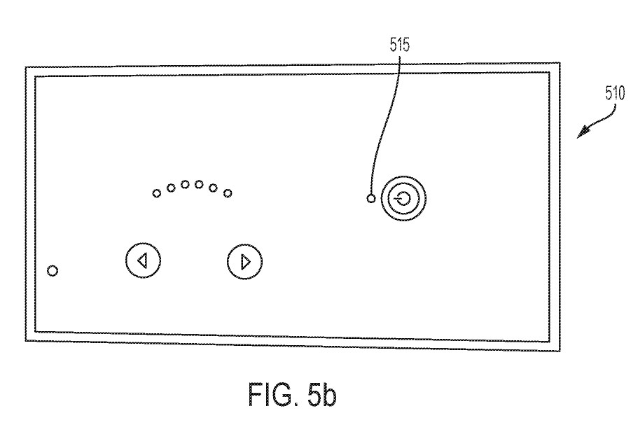

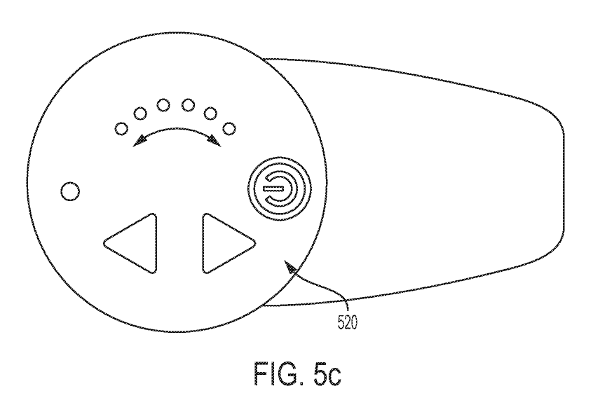

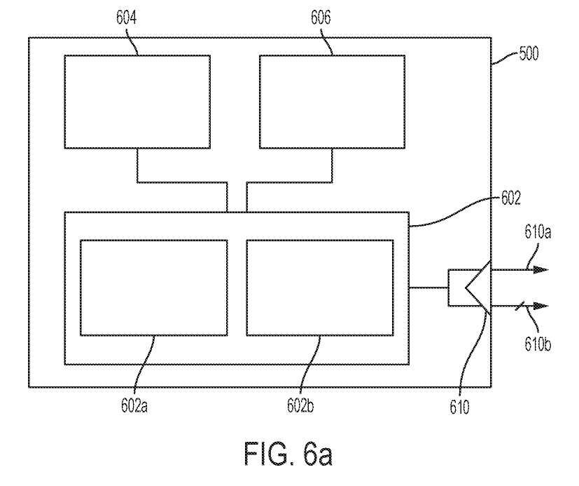

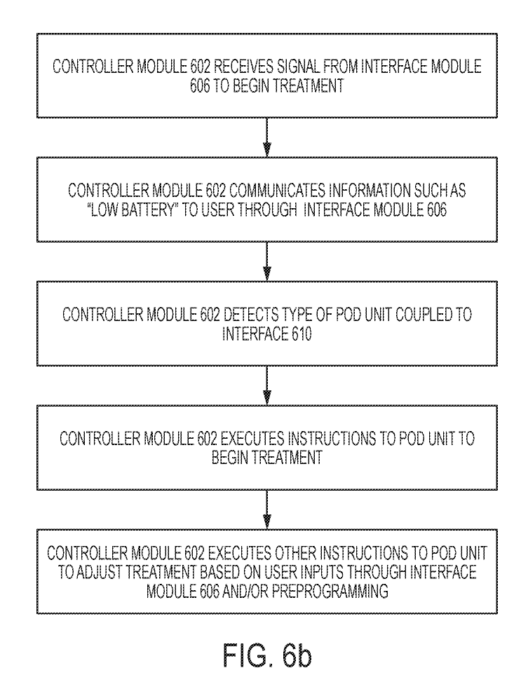

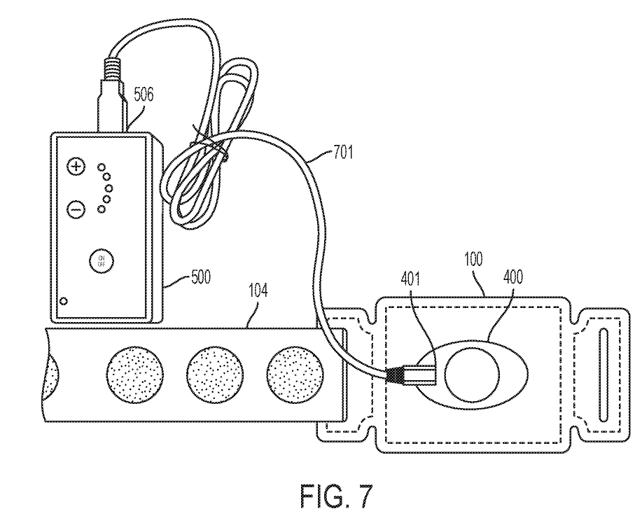

FIG. 5a depicts a perspective view of a controller 500 according to an embodiment of the present invention. Controller 500 can have a shape that fits comfortably within a user's hand and permits the user to push any of the buttons with the hand. In particular, the controller 500 includes an “On/Off” or “Start” button 501 and two intensity-adjustment buttons 502a, 502b. When controller 500 is removably coupled (whether by cable or wirelessly) to a pod unit, such as pod unit 300, positioned for treatment on an area of a user's body, intensity-adjustment button 502a (or “Plus” button) allows a user to increase the intensity of the TENS treatment delivered to the area of the body by a preprogrammed amount each time it is pushed by the user up to maximum programmed amount. Likewise, intensity-adjustment button 502b (or “Minus” button) allows a user to decrease the intensity of the TENS treatment delivered to the area of the body by a preprogrammed amount each time it is pushed by the user down to a minimum programmed amount. Indicator LEDs 503a through 503f indicate to the user the intensity level of the TENS treatment being delivered. In particular, LED 503a is the only LED lit up when the lowest intensity is being delivered; LEDs 503a and 503b are both lit up when the next highest intensity is being delivered (e.g., following a user pushing intensity-adjustment button 502a); LEDs 503a, 503b, and 503c are lit up when the next highest intensity is being delivered (e.g., after a user pushes intensity-adjustment button 502a again); and so on. While six lights are representatively shown, indicating six intensity levels, any number of lights and levels may be utilized. In addition to the six LEDs just described, controller 500 may also include one or more LEDs 504 that can indicate other information, such as when the controller and/or pod unit are on, being charged, when its power (e.g., provided by an internal battery) is low, and/or the type of treatment being delivered. For example, LED 504 may glow green when the controller and/or pod unit are on, yellow when the controller and/or pod unit are charging, and red when the controller and/or pod unit are low on power. Alternatively or additionally, “on/off” button 501 may include an LED that indicates to the user when the controller and/or pod unit are on or other information such that just described. Controller 500 may also include a charging port (not shown) for charging a power storage device, such as a battery, in the controller and/or pod unit, as well as a USB port 506 (see FIG. 7) for connecting controller 500 to a pod unit such as pod unit 400 or 410 via a cable such as cable 701 (see FIG. 7). The electronic functions of controller 500 and interaction with pod unit 400 or 410 are exemplary described with reference to FIGS. 6a-6b below.

FIG. 5b depicts a controller 510 according to an embodiment of the present invention. Controller 510 can function in the same manner as controller 500 with the same components, but includes an additional LED 515 that can indicate information to a user. For example, LED 515 can glow yellow when the internal battery of controller 510 is low or a variety of colors to indicate which type of treatment is being (e.g., blue for TENS and red for LED or Ultrasound).

FIG. 5c depicts a controller 520 according to an embodiment of the present invention. Controller 510 can function in the same manner as controller 500 or controller 510, but has a shape configured to more comfortably fit in a user's hand. For example, controller 520 can have a length of about 12.2 cm and a width of about 6.75 cm at it largest portion.

FIG. 6a is an electronic block diagram of the controller of one embodiment of the present invention, such as controller 500 just described. The controller may include a number of modules 602, 604, and/or 606 for performing certain functions. Certain units described in this specification have been labeled as modules, in order to more particularly emphasize their implementation independence. A module is “[a] self-contained hardware or software component that interacts with a larger system.” Alan Freedman, “The Computer Glossary” 268 (8th ed. 1998). A module comprises a machine or machines executable instructions. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also include software-defined units or instructions, that when executed by a processing machine or device, transform data stored on a data storage device from a first state to a second state. An identified module of executable code may, for instance, comprise one or more physical or logical blocks of computer instructions which may be organized as an object, procedure, or function. Nevertheless, the executables of an identified module need not be physically located together, but may comprise disparate instructions stored in different locations which, when joined logically together, comprise the module, and when executed by the processor, achieve the stated data transformation.

Indeed, a module of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices.

In the following description, numerous specific details are provided, such as examples of programming, software modules, user selections, network transactions, database queries, database structures, hardware modules, hardware circuits, hardware chips, etc., to provide a thorough understanding of the present embodiments. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

Embodiments of the controller 500 may include a controller module 602, a power supply module 604, and/or an interface module 606. Power supply module 604 may include, for example, an energy storage device (e.g., a Lithium-ion or Lithium-polymer battery, a capacitor, a Zinc-air battery, or other type of battery) and circuitry for managing the energy storage device and/or controlling an output power level from the energy storage device. For example, when the storage device is a Lithium-based battery, the power supply module 604 may include circuitry for controlling charging and discharging of the battery and circuitry for regulating a supply voltage generated from the battery. In some embodiments, such circuitry may include a buck converter and/or a high voltage boost converter. A buck converter may be used, for example, to generate a 3.3 Volt power supply for operating logic circuitry within controller 500, such as within controller module 602. A high voltage boost converter may be used, for example, to generate a higher voltage level for operating LEDs, Ultrasound components, TENS components, or other electrical devices requiring a higher voltage level than the cell voltage of the battery. Power supply module 604 may have one or more outputs for different voltage levels, such as a 3.3 Volt output and a high-voltage output. In some embodiments, all outputs may be coupled to controller module 602 for distribution to other modules, such as pod unit 400 or pod unit 410. In other embodiments, some outputs may be coupled to controller module 602 and some outputs may be coupled directly to other components, such as pod unit 400 or pod unit 410.

Controller 500 may also include an interface module 606. Interface module 606 may include, for example, one or more LEDs for providing output to a user, one or more LCDs for providing output and/or receiving input from a user, and/or one or more buttons, switches, toggle levels, or the like, for receiving input from a user. Controller module 602 may be coupled to interface module 606 to control the input from and/or output to a user of the controller 500. For example, interface module 606 may include a “START” or “On/Off” button for activating treatment, and controller module 602 may receive an indication of that input and begin treatment, such as by executing a method described below with reference to FIG. 6b. As another example, interface module 606 may include a series of LEDs to indicate a status of the treatment to the user, and controller module 602 may activate LEDs in the series of LEDs to indicate completion of the various stages of an executed treatment. As a further example, interface module 606 may include a series of LEDs to indicate the intensity of the TENS treatment being delivered and a “Plus” and/or “Minus” button for adjusting the intensity of the delivered TENS treatment, such that controller module 602 may receive an indication of that input, adjust the intensity of the delivered TENS treatment, and communicate the adjustment to the user, such as by executing instructions to interface module 606 to illuminate one or more of the series of LEDs. The interface module may also include one or more LEDs to indicate power status (e.g., on, charging, low battery) of controller 500 and/or pod unit 400 or pod unit 410, and the controller module may activate the one or more LEDs to indicate the status.

Controller module 602 may carry out steps for the operation of controller 500 and/or pod unit 400 or pod unit 410. Controller module 602 may be coupled to an interface 610 for communicating with pod unit 300. Interface 610 may couple to pod unit 400 or pod unit 410 through power lines 610A and data lines 610B. In some embodiments, power lines 610A and data lines 610B may be general purpose conductors that provide both power and data or that are time-shared between power and data. In some embodiments, interface 610 may be a universal serial bus (USB) interface. Controller module 602 may include one or more modules (e.g., LED module 602A and Ultrasound module 602B) for performing certain functions in addition to a central module (not shown) for coordinating between other modules within controller module 602 and other modules in controller 500 and interface 610. LED module 602A may be configured to operate an LED-based pod unit or an LED portion of a pod unit for delivering therapeutic treatment. Ultrasound module 602B may be configured to operate an Ultrasound-based pod unit or Ultrasound portion of a pod unit for delivering therapeutic treatment. Pod unit 300 may be designed for treating a specific area of the body, for example, in any of the embodiments described above, and controller module 602 may be configured to detect the specific pod unit, e.g., pod unit 400 or pod unit 410, coupled to controller 500 by, for example, receiving a signal from the pod unit prior to activating treatment as described below with reference to FIG. 6b. In some embodiments, the signal may include a model number or identifier of the encoded as data and transmitted to controller module 602. In some embodiments, the signal may be used to measure a characteristic of the pod unit that can be used to identify the pod unit, such as an impedance of the connection to the pod unit or a power consumption level of the pod unit. The controller may send different instructions for different therapeutic treatments to the pod unit based on the identity of the pod unit.

FIG. 6b is a flow chart describing operation of the controller when coupled to a pod unit according to one embodiment of the present invention. In operation, a user couples a pod unit, such as pod unit 400 or pod unit 410, to controller 500 through an interface 610. When ready for treatment, the user signals initiation of treatment (e.g., by pushing the “Start” or “On/Off” button). This signal is received by an interface module 606, which communicates it to a controller module 602. Controller module 602 detects the type of pod unit (e.g., for treating the lower back, hip, neck, shoulder, knee, elbow, ankle, wrist, and/or calf) through interface 610 and executes instructions for the pod unit to begin treatment according to the type of pod unit, as illustratively explained above. For example, if the pod unit is configured to treat the elbow of a user, controller module 602 may instruct the pod unit to begin TENS treatment of a mid to high frequency. The pod unit may continue this treatment until it receives new instructions from controller module 602 (e.g., to stop TENS treatment and begin Ultrasound treatment). Controller module 602 may be preprogrammed to execute a specific program for the type of pod unit detected and/or may execute instruction based on user inputs. For example, controller module 602 may receive a user input through a “Plus” or “Minus” button of the interface module 606 to alter the intensity of a delivered TENS treatment and send instructions to the pod unit to adjust the intensity of the delivered TENS treatment based on the input. Controller module 602 may also include limits that restrict the user from increasing the intensity by more than a threshold amount over a specified time period or may also include limits that set a maximum intensity level based on the specific pod unit coupled to controller module 602.

The operations described above as performed by a controller module, when embodied as hardware, may be performed by any circuit configured to perform the described operations. Such a circuit may be an integrated circuit (IC) constructed on a semiconductor substrate and include logic circuitry, such as transistors configured as logic gates, and memory circuitry, such as transistors and capacitors configured as dynamic random access memory (DRAM), electronically programmable read-only memory (EPROM), or other memory devices. The logic circuitry may be configured through hard-wire connections or through programming by instructions contained in firmware. Further, the logic circuity may be configured as a general purpose processor capable of executing instructions contained in software. If implemented in firmware and/or software, functions described above may be stored as one or more instructions or code on a computer-readable medium. Examples include non-transitory computer-readable media encoded with a data structure and computer-readable media encoded with a computer program. Computer-readable media includes physical computer storage media. A storage medium may be any available medium that can be accessed by a computer. By way of example, and not limitation, such computer-readable media can comprise random access memory (RAM), read-only memory (ROM), electrically-erasable programmable read-only memory (EEPROM), compact disc read-only memory (CD-ROM) or other optical disk storage, magnetic disk storage or other magnetic storage devices, or any other medium that can be used to store desired program code in the form of instructions or data structures and that can be accessed by a computer. Disk and disc includes compact discs (CD), laser discs, optical discs, digital versatile discs (DVD), floppy disks and Blu-ray discs. Generally, disks reproduce data magnetically, and discs reproduce data optically. Combinations of the above should also be included within the scope of computer-readable media.

In addition to storage on computer readable medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims.

FIG. 7 depicts a plan view (facing away from a user's skin) of a pod unit 400 removably coupled to a wrap 100 according to one embodiment of the present invention. A strap 104 is removably coupled to one end of wrap 100. A handheld controller 500 is operationally coupled to pod unit 400 via ports 401 and 506 by a cable 701. Cable 701 may be, for example, a male USB Micro to Male USB-A cable or a male-to-male TRRS cable. FIG. 8 depicts a bottom view (facing a user's skin) of pod unit 400 attached to wrap 100 (illustrated without straps) and connected to electrodes 300 by electrode wires 301 according to one embodiment of the present invention. When taken in combination (and with a second strap coupled to the other end of wrap 100), FIGS. 7 and 8 illustrate an example of an electroceutical treatment system ready to be coupled to an area of a user's body for therapeutic treatment.

The above specification and examples provide a complete description of the structure and use of exemplary embodiments. Although certain embodiments have been described with a certain degree of particularity, or with reference to one or more individual embodiments, those skilled in the art could make numerous alterations to the disclosed embodiments without departing from the scope of this invention. As such, the various illustrative embodiments of the present devices are not intended to be limited to the particular forms disclosed. Rather, they include all modifications and alternatives falling with the scope of the claims, and may include some or all of the features of any depicted embodiment. For example, pod unit 300 can have any suitable dimensions or shape that permit the present apparatuses to function as described in this disclosure, e.g., attach to wraps 100 or 110 through hole 101, which may have altered dimensions as well. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

Although the present disclosure and certain representative advantages have been described in detail, it should be understood that various changes, substitutions, and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

The claims are not intended to include, and should not be interpreted to include means-plus or step-plus function limitations, unless such a limitation is explicitly recited in a given claim using the phrase(s) “means for” or “step for,” respectively.

1. An apparatus for delivering therapeutic self-care treatment, comprising:
a controller configured to be coupled to a pod unit for controlling delivery of therapeutic treatment, wherein the controller is configured to perform steps comprising:
receiving an indication to begin the therapeutic treatment;
detecting a type of therapeutic treatment to be delivered;
communicating to the pod unit 15 the type of therapeutic treatment to be delivered,
wherein the pod unit is configured to deliver the therapeutic treatment based on communications received from the controller;
a wrap, wherein the pod unit is configured to be coupled with the wrap; and
an electrode removably coupled to the wrap and configured to be further coupled to the pod unit;
wherein the electrode is not coupled to the wrap, the pod unit, or an area of the user's skin by an adhesive.

2. The apparatus of claim 1, wherein the therapeutic treatment is of fixed frequencies or outputs and delivered in a sequential and timed manner.

3. The apparatus of claim 1, wherein the controller is further configured to perform steps comprising adjusting the therapeutic treatment based on the type of coupled pod unit.

4. The apparatus of claim 3, wherein the therapeutic treatment adjusts between TENS-based and LED- or Ultrasound-based therapeutic treatment.

5. The apparatus of claim 1, wherein the controller is further configured to perform steps comprising communicating the type of therapeutic treatment being delivered to a user.

6. The apparatus of claim 5, wherein the type of therapeutic treatment being delivered is communicated by activating one or more LEDs or by making an audible sound.

7. The apparatus of claim 1, wherein the controller is further configured to perform steps comprising adjusting the intensity of therapeutic treatment based on inputs received from a user.

8. The apparatus of claim 7, wherein the controller is further configured to perform steps comprising communicating the intensity of the therapeutic treatment to the user.

9. The apparatus of claim 8, wherein the intensity of the therapeutic treatment is communicated to the user by activating one or more LEDs or by making an audible sound.

10. The apparatus of claim 1, wherein the controller is configured to couple to the pod unit through a universal serial bus (USB) connection or a wireless connection.

11. (canceled)

12. The apparatus of claim 1, wherein the pod unit is configured to treat the back, hip, neck, shoulder, knee, elbow, ankle, wrist, or calf of the user.

13. The apparatus of claim 1, wherein the pod unit comprises LED-based components or Ultrasound-based components that are configured to provide therapeutic treatment.

14. The apparatus of claim 13, wherein the pod unit further comprises a TENS-based component configured to provide therapeutic treatment.

15. The apparatus of claim 1, wherein the pod unit is configured to provide both TENS-based and LED- or Ultrasound-based therapeutic treatment.

16. (canceled)

17. The apparatus of claim 1, wherein the wrap is adjustable.

18. The apparatus of claim 1, wherein the wrap is configured to be positioned over an area of a user's body underneath the user's clothing.

19. The apparatus of claim 1, further comprising an electrode removably coupled to the wrap and configured to be further coupled to the pod unit, wherein the electrode is not coupled to the wrap, the pod unit, or an area of the user's skin by an adhesive.

20. A method of delivering therapeutic self-care treatment, comprising:
receiving, by a controller, an indication to begin the therapeutic treatment;
detecting, by the controller, a type of therapeutic treatment to be delivered;
communicating, by the controller, to a pod unit the type of therapeutic treatment to be delivered;
coupling the pod unit to a wrap;
coupling the controller to the pod unit through a universal serial bus (USB) connection or a wireless connection; and
removably coupling an electrode to the wrap and the pod unit, wherein the electrode is not coupled to the wrap, the pod unit, or an area of the user's skin by an adhesive.

21-35. (canceled)

36. A therapeutic self-care treatment delivery system, comprising:
a pod unit configured to provide therapeutic treatment;
a controller coupled to the pod unit for controlling delivery of therapeutic treatment by the pod unit, wherein the controller is configured to performs steps comprising:
receiving an indication to begin the therapeutic treatment;
detecting a type of therapeutic treatment to be delivered; and
communicating to the pod unit the type of therapeutic treatment to be delivered;
a wrap, wherein the pod unit is configured to be coupled with the wrap, wherein the wrap is adjustable and wherein the wrap is configured to be positioned over an area of a user's body underneath the user's clothing; and
an electrode removably coupled to the wrap and configured to be further coupled to the pod unit, wherein the electrode is not coupled to the wrap, the pod unit, or an area of the user's skin by an adhesive.

37. The system of claim 36, wherein the therapeutic treatment is of fixed frequencies or outputs and delivered in a sequential and timed manner.

38-54. (canceled)
